(19) United States
(12) Patent Application Publication
(10) Pub. No.: US 2004/0072205 A1
(43) Pub. Date: Apr. 15, 2004

(54) Methods for treating neuronal disorders

(76) Inventor: Robert M. Friedlander, Brookline, MA (US)

Correspondence Address: CLARK & ELBING LLP, 101 FEDERAL STREET, BOSTON, MA, 02110, US

(21) Appl. No.: 10/427,852

(22) Filed: May 1, 2003

Related U.S. Application Data
(60) Provisional application No. 60/376,791, filed on May 1, 2002.

(51) Int. Cl. C12Q 1/68; A61K 31/65

(52) U.S. Cl. 435/6; 514/152

(57) ABSTRACT

The invention features methods for the treatment of neuronal disorders using compounds that reduce or inhibit cytochrome c release. The invention also features methods for identifying compounds that reduce or inhibit cytochrome c release for the treatment of neuronal disorders.

### CROSS REFERENCE TO RELATED APPLICATIONS

This application claims the benefit of the filing date of U.S. Provisional Application No. 60/376,791, filed May 1, 2002.

### FIELD OF THE INVENTION

The invention relates to the treatment of neuronal disorders, specifically amyotrophic lateral sclerosis (ALS) and cerebral ischemia.

### BACKGROUND OF THE INVENTION

Amyotrophic lateral sclerosis (ALS), sometimes called Lou Gehrig's disease, is a rapidly progressive, invariably fatal neurological disease that attacks the neurons responsible for controlling voluntary muscles. The disease belongs to a group of disorders known as motor neuron diseases, which are characterized by the gradual degeneration and death of motor neurons.

Motor neurons are nerve cells located in the brain, brainstem, and spinal cord that connect the nervous system to voluntary muscles of the body. In ALS, the motor neurons degenerate or die, causing the muscles they enervate to gradually weaken, atrophy, and twitch (fasciculation). Eventually, the ability of the brain to control voluntary movement is lost. When muscles in the diaphragm and chest wall fail, patients lose the ability to breathe without ventilatory support, resulting in death due to respiratory failure. This usually occurs within 3 to 5 years from the onset of symptoms.

As many as 20,000 Americans have ALS, and an estimated 5,000 people in the United States are diagnosed with the disease each year. ALS is one of the most common neuromuscular diseases worldwide, and people of all races and ethnic backgrounds are affected. ALS most commonly strikes people between 40 and 60 years of age, but younger and older people can also develop the disease, with men more often affected than women. In 90 to 95 percent of all ALS cases, the disease occurs apparently at random with no clearly associated risk factors. Patients typically do not have a family history of the disease, and their family members are not considered to be at increased risk for developing ALS.

While the cause of ALS is not known, about 20 percent of all cases are familial and result from a specific genetic defect that leads to mutation of the enzyme known as superoxide dismutase 1 (SOD1). SOD1 is a powerful antioxidant that protects the body from damage caused by free radicals produced by cells during normal metabolism. It is not clear how this enzyme is involved in ALS, although transgenic mice expressing several of the mutant SOD1 genes found in humans with ALS develop motor neuron symptoms and histopathology resembling features of the human disease. Other studies on motor neuron degeneration in ALS patients have focused on the role of glutamate, a neurotransmitter found in the brain, autoimmune response against motor neurons, and exposure to toxic or infectious agents.

Current therapy for ALS involves the administration of riluzole, which extends survival in human patients approximately three months. Riluzole is believed to reduce the damage to motor neurons by decreasing the release of glutamate; riluzole does not reverse the damage already done to motor neurons. Because riluzole causes liver damage and has other possible side effects, patients administered the drug must be closely monitored. While current therapies for the treatment of ALS show promise, there exists a need for additional therapies that delay disease onset and extend patient survival.

Minocycline is a second-generation tetracycline that effectively crosses the blood-brain-barrier and demonstrates neuroprotective qualities in experimental models of neurodegeneration, e.g., cerebral ischemia, traumatic brain injury, and Huntington's and Parkinson's disease. Minocycline-mediated neuroprotection is associated with the inhibition of members of the caspase cell death family, in particular caspase-1 and caspase-3, which are known to be activated in neurons of ALS mice. Minocycline-mediated neuroprotection is also associated with the inhibition of the inducible form of nitric oxide synthetase (iNOS), p38 mitogen activated protein kinase (MAPK) activity, and microglial activation. Despite the neuroprotective properties of minocycline, its target is not known.

### SUMMARY OF THE INVENTION

We have discovered that minocycline is an effective therapeutic for the treatment of ALS. We have tested the action of minocycline using cell-free mitochondrial preparations, using cell-based death models, and in vivo using an ALS mouse model. We believe that the neuroprotective effects of minocycline in the ALS model result from the broad inhibition of cell death due to the inhibition of cytochrome c release.

Accordingly, the invention features a method for treating a patient diagnosed with or at risk for developing ALS, involving administering a compound (e.g., minocycline), in an amount sufficient to treat the patient, which reduces or inhibits cytochrome c release by mitochondria in cells of the patient. The amount of minocycline administered can be determined by one skilled in the art, but should be an amount sufficient to reduce or inhibit cytochrome c release by mitochondria in cells of the subject, relative to cytochrome c release by mitochondria in cells of a subject diagnosed with or at risk for developing a neurological disorder (e.g., ALS or cerebral ischemia) when that subject is not treated with the compound.

Desirably, the administration of minocycline results in an increase in survival of motor neuron cells by at least 20%, relative to control cells, and may be more than 50%, 75%, 100%, 200%, or 500%. Comparisons in the survival rate of cells can be made at any time following administration (e.g., one week, one month, three months, six months, one year, five years, etc).

The invention also features a method for identifying a compound that treats or prevents a neuronal disorder (e.g., ALS or cerebral ischemia) the method includes providing a cell-free mitochondrial preparation, a cell (e.g., a neuroblastoma cell), or a non-human mammal (e.g., a mouse), contacting the compound with the preparation, cell, or non-human mammal, and determining whether the compound reduces or prevents cytochrome c release by the mitochondria in the preparation, cell, or in one or more cells of the non-human mammal, relative to cytochrome c release in a preparation, cell, or one or more cells of a non-human mammal not contacted with the compound. In an embodiment, the method further includes, prior to contacting the preparation, cell, or non-human mammal with the compound, contacting the preparation, cell, or non-human mammal with a compound (e.g., calcium or the Bcl-2 interacting protein, Bid) that induces release of cytochrome c from mitochondria. In another embodiment, the genome of the cell or cells of the non-human mammal includes a human SOD1^{G93A} gene.

By “treating” is meant administering a pharmaceutical composition for the treatment or prevention of ALS. To “treat disease” or use for “therapeutic treatment” refers to administering treatment to a patient already suffering from ALS to improve the patient's condition (i.e., to reduce or prevent motor neuron degeneration, preserve motor neuron function, and maintain a patient's normal lifestyle). By “patient” is meant any animal (e.g., a human).

An effective amount of active compound(s) used to practice the present invention for therapeutic treatment of ALS varies depending upon the manner of administration, the age, body weight, and general health of the patient. Ultimately, the attending physician or veterinarian will decide the appropriate amount and dosage regimen. Such amount is referred to as an effective, sufficient, or therapeutic amount.

Other features and advantages of the invention will be apparent from the following detailed description, and from the claims.

### BRIEF DESCRIPTION OF THE DRAWINGS

FIGS. 1A, 1C, and 1D are graphs and FIG. 1B is a table showing that minocycline delays the onset of ALS and extends survival in ALS mice. FIG. 1A: Rotarod evaluation of hSOD1^{G93A} transgenic ALS mice treated with saline (▪) or minocycline (♦; n=10/group, *: P<0.05; error bars represent SEM). FIG. 1B: Table of onset of motor deficits and mortality of ALS mice treated with minocycline or saline. FIGS. 1C and 1D: Cumulative probability of onset of Rotarod deficits (FIG. 1C) and survival (FIG. 1D) in hSOD1^{G93A} mice. Onset of Rotarod deficit and mortality were significantly delayed in ALS mice treated with minocycline compared to mice treated with saline. Solid line, minocycline treated ALS mice; dashed line, saline control ALS mice.

FIGS. 2A-2C are graphs showing that minocycline inhibits cell death, caspase activation, and cytochrome c release. FIGS. 2D and 2E are photographs showing western blots of SH-SY5Y neuroblastoma cells exposed to THG. FIG. 2A: Rat primary cortical neurons were exposed to N-methyl-D-aspartate (NMDA) in the presence or absence of minocycline and cell death was assessed by measuring lactate dehydrogenase (LDH) release. Minocycline inhibited NMDA-induced cell death. FIGS. 2B and 2C: Assessment of H₂O₂-mediated (FIG. 2B) and thapsigargan (THG)-mediated (FIG. 2C) SH-SY5Y neuroblastoma cell death and its inhibition by minocycline. FIG. 2D: Western blot of caspase-9 and caspase-3 in lysates of SH-SY5Y neuroblastoma cells exposed to THG. Minocycline-mediated inhibition of THG-induced cell death correlates with inhibition of caspase-9 and caspase-3 activation. FIG. 2E: Western blot of cytochrome c in the cytosolic fraction (10 μg) of SHSY5Y neuroblastoma cells exposed to THG. Minocycline inhibits cytochrome c release in response to THG. In cell death assays, data are representative of three independent experiments. (*: P<0.05; **: P<0.01; error bars represent SEM).

FIGS. 3A-3C are photographs of western blots showing that minocycline inhibits cytochrome c release and swelling in purified nonsynaptosomal brain mitochondria. FIGS. 3D-3E are graphs showing that minocycline alters mitochondrial membrane potential and transmittance. FIGS. 3A and 3B: Minocycline inhibited both calcium- and Bid-induced release of cytochrome c from purified mouse liver mitochondria. Mouse liver mitochondria (0.5 mg/ml) were incubated with minocycline or CsA and cytochrome c release was induced by CaCl₂ (FIG. 3A) or purified Bid protein (FIG. 3B). T=total mitochondrial cytochrome c. COX IV was used as a loading control. FIG. 3C: Cytochrome c release in rat non-synaptosomal brain mitochondria (1 mg/ml) exposed to 40 μM Ca²⁺ in the presence of varying minocycline concentrations. FIGS. 3D and 3E: The change in membrane potential (Δψ; FIG. 3d) and light transmittance (FIG. 3E) in rat non-synaptosomal brain mitochondria (1 mg/ml) exposed to 40 μM Ca²⁺ in the presence of varying minocycline concentrations. Insert in FIG. 3E: mean±SD of the swelling (increase in transmittance) relative to control at 600 seconds (shown as 100%). Bars colored as main key, * P<0.01 vs 0, 10 and 100 μM minocycline, ANOVA followed by the statistically conservative Tukey/Kramer post-hoc. FIG. 3F: Representative traces of effects of minocycline on swelling under conditions associated with PT induction in diluted rat liver mitochondria (0.03 mg/ml). Initial transmittance (T %) normalized to start at a common point (mean adjustment <1% of initial value).

FIGS. 4A, 4C, and 4D are photographs and FIGS. 4B and 4E are graphs showing the effect of minocycline on cytochrome c release and caspase activation. FIG. 4A is a photograph of a western blot showing that minocycline inhibits cytochrome c release in ALS. ALS mice were injected with minocycline for 10 days starting at eight weeks of age. Spinal cord cytosolic components (40 μg) were evaluated by western blot for cytochrome c release. FIG. 4B is a graph showing that minocycline inhibits cytochrome c release in ALS mice injected with minocycline for 10 days starting at eight weeks of age. Densitometric quantification of cytochrome c release (n=5, *: P<0.05). FIG. 4C is a photograph of a western blot of a spinal cord lysate from ALS mice treated with and without minocycline showing caspase-3 using an antibody specific for activated caspase-3. Minocycline inhibits caspase-3 activation in spinal cords of ALS mice. FIG. 4D is a photograph of a western blot of cytosolic components (cytochrome c and tubulin) of the ischemic territory of mouse brains that were fractionated and analyzed for cytochrome c release. Minocycline inhibits cerebral ischemia-mediated cytochrome c release. FIG. 4E is a graph showing densitometric quantification of cytochrome c release in ischemic mouse brain tissue (n=5, **: P<0.01).

### DETAILED DESCRIPTION

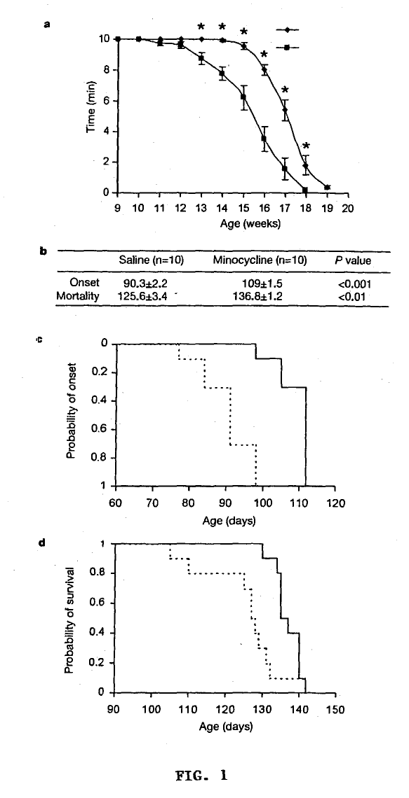

We tested the effectiveness of minocycline on a mouse model of ALS and investigated its mechanism of neuroprotection. Minocycline delays disease onset and extends survival in ALS mice. We evaluated the effect of minocycline on one of the ALS mouse models expressing the mutant human SOD1^{G93A} transgene. Minocycline (10 mg/kg body weight/day) was injected beginning at five weeks of age. Mutant hSOD1^{G93A} transgenic mice were evaluated weekly on a Rotarod to follow disease progression. Onset of impaired motor performance in minocycline-treated mice was delayed to 109.0±1.5 days of age as compared to 90.3±2.2 days in saline-treated mice (P<0.001; FIG. 1A). Minocycline administration extended survival from 125.6±3.4 days to 136.8±1.2 days (P<0.01; FIGS. 1B-1D).

Minocycline-mediated neuroprotection in hSOD1^{G93A} mice confirmed using a slightly modified protocol. Minocycline administration started at six weeks of age at a slightly higher dose (11 mg/kg body weight/day). Mortality of saline-treated mice was 126.3±2.7 days as compared to 139.0±2.1 days of the minocycline-treated mice (n=8/group, P<0.05). These results demonstrate the effectiveness of minocycline in this transgenic ALS model.

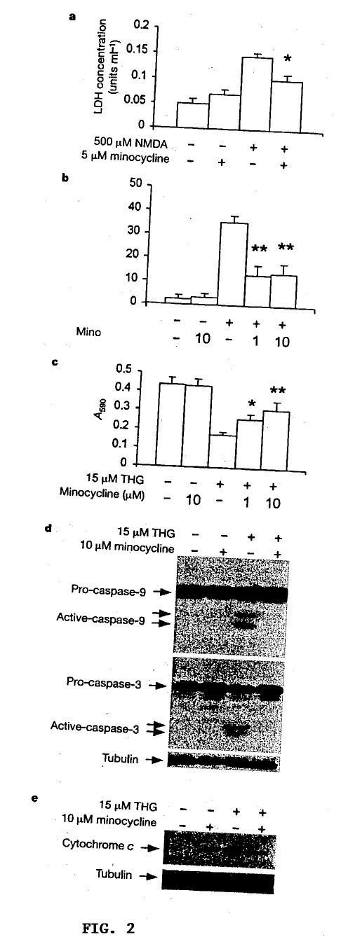

We then investigated the mechanism of minocycline-mediated neuroprotection. Among the known activities of minocycline include inhibition of caspase-1 and caspase-3 transcriptional upregulation and activation. Minocycline does not directly inhibit caspase-1 or casapse-3 activity (Chen et al., Nat. Med. 6:797-801, 2000). A challenge of elucidating the primary neuroprotective target of minocycline in vivo, especially in chronic neurodegenerative diseases, is that many pathways become activated during the progression of the disease as a result of a secondary reactive process. To determine the primary target/targets of minocycline, we searched for in vitro models where cell death is inhibited by minocycline. Since minocycline mediates remarkable neuroprotection in ischemia and traumatic brain injury, we evaluated its effect on N-methyl-D-aspartate (NMDA)-mediated cell death in primary cerebrocortical neurons (Yrjanheikki et al., Proc. Natl. Acad. Sci. U.S.A. 95:15769-15774, 1998; Sanchez et al., Neurosurgery 48:1399-1401, 2001). Minocycline inhibited NMDA-induced cell death (FIG. 2A). In addition, minocycline inhibited death of SH-SY5Y neuroblastoma cells treated with either H₂O₂ or Thapsigargin (THG; FIGS. 2B and 2C). Similar to its broad in vivo neuroprotective properties, minocycline inhibits cell death in a variety of in vitro models of cell death, suggesting that minocycline inhibits a critical shared step in the execution of the death program. Since minocycline does not directly inhibit caspase-1 or caspase-3, we evaluated its effect on caspase-9 activation and cytochrome c release (Chen et al., supra). Minocycline-mediated inhibition of THG-induced cell death correlates with the inhibition of caspase-9 and of caspase-3 activation, as well as of cytochrome c release (FIGS. 2D and 2E).

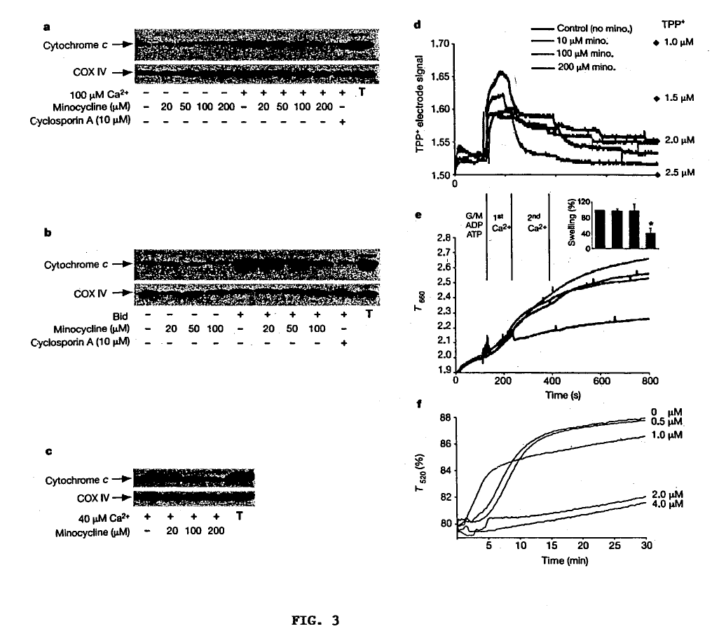

Cytochrome c release from the mitochondria into the cytoplasm is a potent physiologic stimulus for caspase-9 and caspase-3 activation (Green and Reed, Science 281:1309-1312, 1988). Therefore, we tested the effect of minocycline on cytochrome c release using cell-free mitochondrial preparations. Minocycline inhibited both calcium- and Bid-induced cytochrome c release in purified mouse liver mitochondria (FIG. 3A; Li et al., Cell 94:491-501, 1998; Luo et al., Cell 94:481-490, 1998). Due to the critical role of mitochondria in apoptotic pathways, the relation between minocycline and inhibition of cytochrome c release is likely an important clue as to one of its primary/direct mechanisms of action (Green et al., supra).

To confirm that mitochondria are direct targets of minocycline in the brain, and further probe into its mechanism of action, we evaluated the effect of minocycline on isolated nonsynaptosomal brain mitochondria. Cytochrome c release, changes in mitochondrial membrane potential (Δψ), Ca²⁺-transport, oxygen consumption and transmittance at 660 nm were monitored following Ca²⁺ addition (FIGS. 3C-3E). As demonstrated in isolated liver mitochondria, and in the above-described models, minocycline inhibited cytochrome c release in isolated brain mitochondria. Minocycline addition was associated with alteration in several mitochondrial parameters, such as the decrease in Δψ (FIG. 3D), but the change in transmittance (FIG. 3E) was the only change whose dose-specific effect reflected that of inhibition of cytochrome c release. Specifically, minocycline prevented the majority of the increase in transmittance that begins immediately upon Ca²⁺ addition. These studies conducted in concentrated mitochondrial solutions appropriate for biochemical studies required 200 μM minocycline for complete protection (FIGS. 3A-3E). Expressing minocycline levels as concentrations, though, overstates the requirements if minocycline is accumulated or acts stoichiometrically, in which case minocycline levels are more appropriately expressed in mol/mg protein. Co-titration of minocycline and mitochondria demonstrated minocycline-mediated inhibition of mitochondrial swelling at physiologically relevant concentrations of 2-4 μM in isolated rat liver mitochondria (≧40 nmol/mg protein, FIG. 3F). Traces are representative of a total of 24 experiments (3 independent mitochondrial preparations, 2 mitochondrial concentrations [0.1 mg/ml, 0.03 mg/ml], 2 substrates [glutamate/malate, succinate], 2 duplicates) on minocycline-mediated protection. For 0.1 mg/ml protein, 12 of 12 samples were protected by 4 μM minocycline (40 nmol/mg protein); for 0.03 mg/ml protein, 12 of 12 samples were protected by 2 μM minocycline (66 nmol/mg protein). The described minocycline-mediated effects were highly reproducible and statistically significant (p<0.0001 ANOVA followed by the Fischer PLSD post-hoc test).

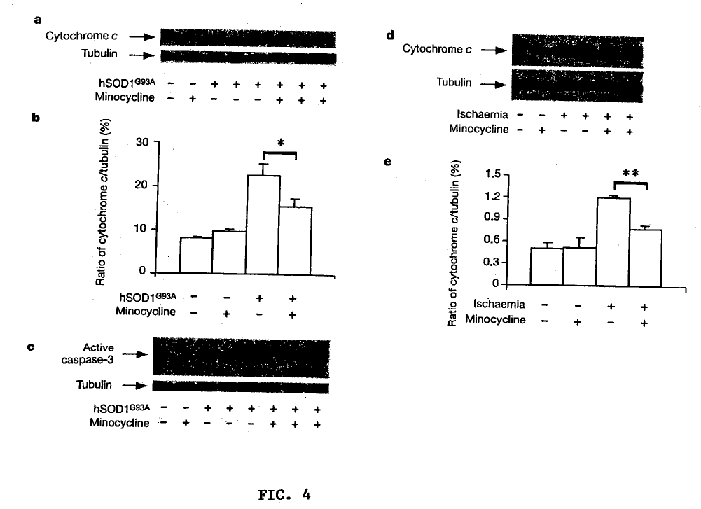

To confirm the findings of minocycline-mediated inhibition of cytochrome c release in vivo, we evaluated whether minocycline-mediated neuroprotection in ALS mice is associated with inhibition of cytochrome c release. Consistent with a previous report, release of cytochrome c in spinal cords of ALS mice correlates with disease progression (Guegan et al., J. Neuroscience 21:6569-6576, 2001). Minocycline treatment significantly inhibited release of cytochrome c in ALS mice (n=5, P<0.05; FIGS. 4A and 4B). As a confirmatory marker of efficacy, we demonstrated that minocycline inhibits caspase-3 activation in spinal cords of ALS mice (FIG. 4C). To extend these findings, we demonstrated that minocycline inhibits cerebral ischemia-mediated cytochrome c release (n=5, P<0.01; FIGS. 4D and 4E). These results provide in vivo confirmation of the effect of minocycline on cytochrome c release and support the finding that minocycline inhibits cytochrome c release in cells and in isolated mitochondria, and can be used to treat ALS and cerebral ischemia.

The data obtained is most consistent with a model where minocycline acts directly on the mitochondria to alter PT-mediated cytochrome c release. While other possibilities cannot be ruled out, several lines of evidence point to involvement of PT in the effects demonstrated by minocycline. Release of cytochrome c from isolated liver mitochondria occurs following exposure to exogenous Ca²⁺, the standard PT challenge, or Bid, which has been shown to accelerate PT induction, and is prevented in both cases by cyclosporin (CsA), the standard PT inhibitor (Zamzami et al., Oncogene 19:6342-6350, 2000; Bernardi et al., Eur. J. Biochem. 264:687-701, 1999). In non-synaptosomal mitochondria, minocycline failed to significantly block Ca²⁺-cycling driven loss of Δψ (FIG. 3D), but did consistently prevent over 50% of the total change in transmittance (FIG. 3E). Transmittance is the physical equivalent of the absorbance change considered a standard hallmark of PT induction in liver mitochondria and more controversially, one measure of “PT-like” behavior in nonsynaptosomal preparations (Kristal and Dubinsky, J. Neurochem. 69:524-538, 1997; Friberg et al., J. Neurochem. 72:2488-2497, 1999). Given that the exact mechanism mediating cytochrome c release remains unclear and controversial, these results do not rule out the possibility that alternative PT-independent pathways might exist resulting in cytochrome c release (Green and Reed, supra; Bernardi et al., supra; Shimizu et al., Nature 399:483-487, 1999).

We validated the mechanism of action of minocycline at three complimentary levels: using cell-free mitochondrial preparations, using cell based death models, and in vivo using ALS mice and cerebral ischemia models. Since cytochrome c release is a shared feature of many neurologic disorders, inhibition of PT-mediated cytochrome c release by minocycline explains the broad inhibition of cell death both in vivo and in vitro mediated by this drug. We have shown that minocycline, which is a non-toxic drug with a proven human safety record, inhibits cytochrome c release and, given its safety in chronic diseases, its oral bioavailability, and its ability to cross the blood-brain-barrier, can be used to treat pathophysiological conditions, such as ALS and cerebral ischemia in humans (Brogden et al., Drugs 9:251-291, 1975).

Methods

Minocycline Treatment of ALS Mice

ALS mice (Jackson Laboratories, Bar Harbor, Me.) were injected intraperitoneally daily with saline or minocycline (Sigma, St. Louis, Mo.). Strength and coordination were evaluated weekly by Rotarod (Columbus Instruments, Columbus, Ohio). Disease onset was defined as the first day a mouse could not remain on the Rotarod for 10 min at 15 rpm. Mortality was scored as age of death or age when the mouse was unable to right itself within 30 seconds (Li et al., supra).

NMDA Neurotoxicity

Primary cortical neurons isolated from E16 rats and cultured as described (Matsumoto et al., J. Cereb. Blood Flow Metab. 19:736-741, 1999). After two weeks, cultures were exposed for six hours to 500 μM NMDA (Sigma) with or without a two-hour minocycline preincubation. Cytotoxicity was assayed by measuring LDH release (Matsumoto et al., supra).

Minocycline Inhibition of SH-SY5Y Cell Death

Human neuroblastoma SH-SY5Y cells were preincubated with media containing minocycline for 24 hours at 37° C., and later exposed to 6 mM H₂O₂ for 4 hours at 37° C. Cells were then incubated with calcein-AM (1 μM, Molecular Probes, Eugene, Oreg.) in PBS for 40 minutes at 37° C. Cell Viability was determined using an LJL 96, 384 Analyst (Molecular Devices, Sunnyvale, Calif.) fluorescence reader. Viability is converted to cell death ratio in the figure. For THG experiments, cells were preincubated with minocycline for 1 hour and then exposed to 15 μM THG (Sigma). After 12 hours, cell death was evaluated by MTT assay (Roche, Mannheim, Germany).

Middle Cerebral Artery (MCA) Occlusion

C57/B6 mice (18-20 g) were injected with minocycline at 45-mg/kg body weight 4 hours before ischemia, then at 22.5 mg/kg body weight twice a day (Yrjanheikki et al., supra). After 120 min of MCA occlusion, blood flow was restored. Brains were removed after 24 hours and the ischemic territory dissected for cytochrome c release evaluation.

Tissue and Cell Cytosolic Fractionation

Brains or spinal cord samples were homogenized (10 mM Hepes, pH 7.4, 250 mM sucrose, 10 mM KCl, 1.5 mM MgCl₂, 1 mM EDTA, 1 mM EGTA, 1 mM DTT, 50 μM zVAD) plus protease inhibitor cocktail (Roche) in a Kontes dounce homogenizer with a B pestle (Kontes Glass Company, Vineland, N.J.). Cytosolic component was fractionated as previously described (Martin, J. Neuropathol. Exp. Neurol. 58:459-471, 1999). SH-SY5Y cell cytosolic fraction was prepared 6 hours after THG treatment using the same protocol as for mouse brains.

Western Blot

For caspase activation, samples were lysed in RIPA buffer with protease inhibitors. For cytochrome c release, cytosolic component from tissue (40 μg) or cells (10 μg) were loaded for evaluation. Cytochrome c, caspase-9, and caspase-3 antibodies were purchased from PharMingen, San Diego, Calif.; COX IV antibody from Clontech, Palo Alto, Calif.

Liver and Brain Mitochondria Preparation

Mouse liver mitochondria were prepared as described and resuspended in MRM buffer (250 mM sucrose, 10 mM Hepes, pH 7.5, 1 mM ATP, 5 mM sodium succinate, 80 μM ADP, 2 mM K₂HPO₄,) at a concentration of 0.5 mg/ml (Li et al., supra).

Rat liver mitochondria were isolated from 4-6 month old Fischer 344× Brown Norway F₁ rats by differential centrifugation as previously described, except the final wash and resuspension buffer had no EGTA, EDTA, or BSA (Carri et al., FEBS Lett. 414:365-368, 1997).

Non-synaptosomal rat brain mitochondria were prepared from forebrains of ˜8 week Fischer 344× Brown Norway rats (Ficoll gradient purification; Lai et al., Methods Enzymol. 55:51-60, 1979; Kristal and Shestopalov, Dev. Neurosci. 22:376-383, 2000).

In Vitro Cytochrome c Release and Evaluation of Mitochondria Physiologic Parameters

An aliquot of 50 μl of (0.5 mg/ml) mouse liver mitochondrial preparation was preincubated with minocycline or cyclosporin A (10 μM) for 5 min in MRM buffer. Mitochondria were incubated with 100 μM CaCl₂ or 100 ng of purified mouse Bid protein (Li et al., supra) at 30° C. for 30 min (CaCl₂) or for one hour (Bid). Mixtures were centrifuged at 10,000 g at 4° C. for 10 min and the supernatant evaluated by Western blot.

Δψ/Swelling Measurements

Rat brain mitochondria (1 mg/ml in 100 mM KCl, 75 mM mannitol, 25 mM sucrose, 10 mM Tris, 1 mM K—PO₄ (pH 7.3) containing TPP+, 25 μM Ca²⁺ (from buffer and TPP+stock) and minocycline (see key). Mitochondria were added at t=0 and energized where marked with 5 mM glutamate, 5 mM malate, 1 mM ATP and 80 μM ADP. Bolus doses of 40 μM Ca²⁺ were added where shown. Simultaneous measurement of Δψ and light transmittance in stirred samples was accomplished using a four-channel respiration system designed by Dr. Boris Krasnikov (Krasnikov et al., FEBS Lett. 419:137-140, 1997). Oxygen uptake, Δψ, and Ca²⁺ were measured using Clark, TPP+, and Ca²⁺-sensitive electrodes, respectively. A₆₆₀ was measured using a diode. Experiments were carried out in triplicate. Plots shown are representative. Mixtures were sampled and centrifuged at 10,000 g at 4° C. for 10 min. Supernatants were evaluated for cytochrome c.

Rat liver mitochondria were used for minocycline-mitochondria co-titration. PT induction was assessed spectrophotometrically by suspending mitochondria at 25° C. in 200 μl of 310 mM sucrose, 30 mM KCl, 3 mM K-Hepes (pH 7.3), with 5 μM added Ca²⁺. Samples with 0.1 mg/ml mitochondria had 100 μM KPO₄; samples with 0.03 mg/ml mitochondria had 30 μM KPO₄. Changes in transmittance at 520 nm were followed for 30 minutes using a SpectraMax 250 Plate Reader (Molecular Dynamics). Minocycline does not display appreciable absorbance at this wavelength.

Light scattering data is qualitatively identical at the two wavelengths used. The lower wavelength (520 nm) was used on the plate reader because it gives a slightly better signal:noise profile. The higher wavelength (660 nm) was used in the four-channel system because the light emitting diode can only be used at that wavelength.

All publications and patent applications mentioned in this specification are herein incorporated by reference to the same extent as if each independent publication or patent application was specifically and individually indicated to be incorporated by reference.

What is claimed is:

1. A method for treating amyotrophic lateral sclerosis (ALS) in a subject in need thereof, said method comprising administering to said subject a therapeutically effective amount of a compound that reduces cytochrome c release by cells of said subject.

2. The method of claim 1, wherein said compound is minocycline.

3. A method for treating cerebral ischemia in a subject in need thereof, said method comprising administering to said subject a therapeutically effective amount of a compound that reduces or inhibits cytochrome c release by cells of said subject.

4. The method of claim 3, wherein said compound comprises minocycline.

5. A method of determining whether a test substance has the potential to treat a neuronal disorder, said method comprising the steps of:
a) providing a cell-free mitochondrial preparation;
b) contacting said test substance with said mitochondrial preparation; and
c) determining whether said test substance induces a change in a property of said cell-free mitochondrial preparation.

6. The method of claim 5, wherein said property is the reduction or inhibition of cytochrome c release.

7. The method of claim 5, wherein the neuronal disorder is ALS or cerebral ischemia.

8. The method of claim 5, wherein said mitochondria, prior to or during step b), are stimulated with a compound that induces release of cytochrome c from mitochondria.

9. The method of claim 8, wherein said compound is calcium or Bid.

10. A method of determining whether a test substance has the potential to treat a neuronal disorder, said method comprising the steps of:
a) providing a cell;
b) contacting said cell with said test substance; and
c) determining whether said test substance reduces or inhibits cytochrome c release by mitochondria of said cell, relative to cytochrome c release by mitochondria of a cell not contacted by said test substance.

11. The method of claim 10, wherein said cell is a neuroblastoma cell.

12. The method of claim 10, wherein the genome of said cell comprises an human SOD1^{G93A} gene.

13. The method of claim 10, wherein said neuronal disorder is ALS or cerebral ischemia.

14. The method of claim 10, wherein said cell, prior to or during step b), is stimulated with a compound that induces release of cytochrome c from mitochondria.

15. The method of claim 14, wherein said compound is calcium or Bid.

16. A method of determining whether a test substance has the potential to treat a neuronal disorder, said method comprising the steps of:
a) providing a non-human mammal;
b) administering said test substance to said non-human mammal; and
c) determining whether said test substance reduces or prevents cytochrome c release by mitochondria in cells of said non-human mammal, relative to cytochrome c release by mitochondria in cells of a non-human mammal not administered said test substance.

17. The method of claim 16, wherein the genome of said non-human mammal comprises an human SOD1^{G93A} gene.

18. The method of claim 16, wherein said non-human mammal is a mouse.

19. The method of claim 16, wherein said neuronal disorder is ALS or cerebral ischemia.

20. The method of claim 16, wherein said non-human mammal, prior to or during step b), is administered a compound that induces release of cytochrome c from mitochondria.

21. The method of claim 20, wherein said compound is calcium or Bid.
